(19) United States
(12) Patent Application Publication
(10) Pub. No.: US 2016/0222432 A1
(43) Pub. Date: Aug. 4, 2016

(54) TEST STRIP USING FORMALDEHYDE OR PEROXIDE, FROM AMONG SARCOSINE METABOLITES, FOR DIAGNOSING PROSTATE CANCER, AND METHOD FOR DIAGNOSING PROSTATE CANCER USING SAME

(71) Applicant: CSQUARE CO., LTD, Gangwon-do (KR)

(72) Inventor: Youngsoo LEE, Chuncheon-si, Gangwon-do (KR)

(73) Assignee: CSQUARE CO., LTD, Chuncheon-si, Gangwon-do (KR)

(21) Appl. No.: 15/022,102

(22) PCT Filed: Sep. 30, 2014
(86) PCT No.: PCT/KR2014/009215

§ 371 Date: Mar. 15, 2016

(30) Foreign Application Priority Data
Oct. 4, 2013 (KR) 10-2013-0118446
Oct. 11, 2013 (KR) 10-2013-0121327

(51) Int. Cl. C12Q 1/26; C12Q 1/28

(52) U.S. Cl. CPC C12Q 1/26; C12Q 1/28; G01N 2333/90683; G01N 2333/908

(57) ABSTRACT

The present invention relates to a test strip using formaldehyde or peroxide, which are sarcosine metabolites, for diagnosing prostate cancer, and a method for diagnosing prostate cancer by means of the test strip, the present invention allowing a simpler and more rapid diagnosis of prostate cancer than the conventional methods, such as using a spectrophotometer, and with respect to qualifying and quantifying sarcosine in urine, the test strip is reacted in a test urine sample, as with a conventional urinalysis test strip, so as to allow the color change to be visually checked, thereby allowing prostate cancer to be diagnosed without professional personnel and expensive equipment.

### TECHNICAL FIELD

The present invention relates to a prostate cancer diagnostic test strip and a method of diagnosing prostate cancer using the same, and specifically, to a test strip for diagnosing prostate cancer using formaldehyde or a peroxide that is produced when sarcosine contained in urine of a prostate cancer patient is oxidized, and a method of diagnosing prostate cancer using the same.

### BACKGROUND ART

According to the World Health Organization (WHO), prostate cancer is the sixth most common cancer in males, following lung cancer, stomach cancer, liver cancer, colon cancer, and esophageal cancer. In the USA, about 186,000 patients are diagnosed with prostate cancer every year, which results in death in about 29,000. Prostate cancer is one of the most common cancers among men. However, it is difficult to track the onset of the disease. As a general diagnostic method, a blood test for detecting a specific protein called a prostate-specific antigen (PSA) in blood and a follow up biopsy are used.

However, in the related art, a level of the PSA becomes higher in prostate cancer in some cases, which does not directly indicate the presence of a tumor. A high level of the PSA does not indicate malignancy thereof. Also, a small amount of the PSA is contained in blood of healthy males, which causes prostate cancer diagnosis using a PSA test to be less reliable.

Recently, Dr. Arul M. Chinnaiyan (University of Michigan Medical School) has reported in Nature that it is possible to determine prostate cancer and a metastasis tendency in other organs according to an amount of the metabolite sarcosine (produced in prostate cancer cells) detected in urine.

Researchers detected prostate cancer in urine of prostate cancer patients at an early stage, a progressive stage, and a metastatic stage, and confirmed a compound that can be used to identify benign cancer and the form of invasiveness in urine. Also, they announced the fact that a concentration of sarcosine, which is a methylated form of the amino acid glycine, is increased in urine of prostate cancer patients, and particularly, the concentration of sarcosine becomes higher when cancer cells are highly likely to spread to other organs. They announced the fact that a level of sarcosine is more accurate as a diagnostic indicator than the PSA that is generally used to diagnose prostate cancer in the related art.

Various attempts to develop a method of detecting sarcosine in urine using the same and diagnostic indicator applications of prostate cancer are being conducted.

In the related art, an HPLC method and a method in which a urine specimen is treated with a sarcosine oxidase and then a spectrophotometer is used are generally used. In both methods, quantification is possible. However, there are problems in that a professional investigator with expertise needs to use an expensive device, a sample needs to be subjected to complex processes to be performed through several steps, and thus uses thereof are limited.

Several prior arts related to prostate cancer diagnosis techniques and methods thereof are as follows.

The invention of a patent application (No. 2012-529021) filed in Japan on Jun. 2, 2010, by Charité—Universitätsmedizin Berlin relates to a method of diagnosing prostate cancer or a predisposition thereto ex vivo. The method includes a step in which at least one metabolite of a test sample of a subject affected with prostate cancer or suspected of having a predisposition thereto is measured and a step in which the at least one metabolite is used to diagnose prostate cancer or a predisposition thereto. The prior invention includes a collection of metabolites, a collection of data including characteristic values of the metabolites, and a storage medium including the data collection. The prior invention also provides a system connected in a form that can be operated with a data storage medium and configured to compare characteristic values of metabolites of the sample. Also, the prior invention includes a diagnosis technique using at least one metabolite and use of one metabolite for providing a diagnosis technique for diagnosing prostate cancer.

The above prior invention relates to a method of classifying metabolites related to prostate cancer. Measurement of a content of the sarcosine for diagnosing prostate cancer is described in the invention.

In the prior invention, in order to measure a content of the sarcosine in urine, known methods, for example, liquid chromatography (LC) mass spectrometry and/or gas chromatography (GC) mass spectrometry for directly measuring a content of the sarcosine in urine, are used.

On the other hand, the present invention is different from the prior invention in that a prostate cancer diagnostic test strip using formaldehyde, which is a sarcosine metabolite, and a method of diagnosing prostate cancer using the same are provided.

In addition, an invention of a patent “Colorimetric measurement method of specimen according to enzymatic oxidation and reagent,” (Korea Patent Application No. 1992-0001449) applied for in Korea on Aug. 9, 1989 and granted to Boehringer Ingelheim GmbH relates to a colorimetric measurement method of a specimen according to enzymatic oxidation of a specimen. In colorimetric measurement of a specimen according to enzymatic oxidation of a specimen in the presence of an electron acceptor, and a method of measuring a reduced electron acceptor using color development as a measurement value of an amount of the specimen, in the presence of a substance selected from among groups including compounds having nitrogen in an oxidation step between +1 and −1 as a direct electron acceptor, the specimen is oxidized by an appropriate oxidoreductase.

Compared to the present invention in which a sarcosine oxidase is used, in the prior invention, a glucose oxidoreductase is used, and a colorimetric measurement method for measuring an amount of the specimen is described. However, the prior invention is primarily different from the present invention in that no sarcosine metabolite is used.

In addition, an invention “Method of diagnosing and treating preeclampsia or eclampsia,” (Korea Patent Application No. 2007-0001991) filed in Korea on Sep. 1, 2006 by Beth Israel Deaconess Medical Center relates to a method of treating preeclampsia or eclampsia using a compound used to increase a concentration of VEGF or P1GF or using a compound used to decrease a concentration of sF1t-1. Also, in the method, a concentration of sF1t-1, VEGF, or P1GF is detected to monitor treatment of preeclampsia or eclampsia. Also, in the method, a concentration of sF1t-1, VEGF, or P1GF of a subject is detected to diagnose preeclampsia or eclampsia.

In the prior invention, a configuration in which a concentration of VEGF or P1GF in a urine sample is detected to diagnose preeclampsia or eclampsia is described. On the other hand, the present invention is different from the prior invention in that a configuration in which a sarcosine oxidase is used to detect a content of the sarcosine in order to diagnose prostate cancer is provided. The two inventions are partially similar to each other since both use a colorimetric test strip method in order to diagnose a disease. However, the present invention is different from the prior invention in use of a colorimetric test strip method in which formaldehyde produced through oxidation of sarcosine is detected.

In addition, an invention “Metabolic profiling method of prostate cancer,” (Japanese Patent Application No. 2010-537170) filed on Aug. 15, 2008 by Metaboton, Inc. relates to a cancer marker, and particularly, to an abnormal metabolite in prostate cancer, and diagnosis, research, and treatment uses in which a cancer-specific metabolite is used as a target.

The prior invention is partially similar to the present invention in that a content of the sarcosine is measured in order to diagnose prostate cancer. However, the prior invention is different from the present invention in which use of a sarcosine oxidase for measuring a content of the sarcosine is not described. In the present invention, a colorimetric test strip method is used to detect formaldehyde produced when sarcosine is oxidized, instead of using gas chromatography-mass spectrometry (GC-MS) and ultra high performance liquid chromatography-mass spectrometry (UHPLC-MS) in order to measure a content of the sarcosine in urine.

### DISCLOSURE

### Technical Problem

The present invention has been made in view of the above-described problems. The present invention provides a test strip and a method thereof in which, when sarcosine in urine is qualified and quantified, a test strip is reacted with a urine specimen like a urine test strip of the related art, and a change in color can be identified with the naked eye. Therefore, it is possible to diagnose prostate cancer without professional staff or an expensive device.

### Technical Solution

In order to achieve the above-described objects, according to an aspect of the present invention, there are provided a prostate cancer diagnostic test strip using formaldehyde, which is a sarcosine metabolite, a method of producing the same, and a method of diagnosing prostate cancer using the same. There is provided a prostate cancer diagnostic test strip, including: a sarcosine oxidase producing sarcosine metabolites; and 4-amino-3-hydrazino-5-mercapto-1,2,4-triazole (AHMT) that is reacted with formaldehyde produced when sarcosine is oxidized and serves as a chromogen.

There is provided a prostate cancer diagnostic test strip including: a sarcosine oxidase producing sarcosine metabolites; and N-methylbenzothiazolinone-2-hydrazone (MBTH) that is reacted with formaldehyde produced when sarcosine is oxidized and serves as a chromogen.

There is provided a method of producing a prostate cancer diagnostic test strip including: immersing a cellulose test strip in a solution containing a sarcosine oxidase producing sarcosine metabolites, a buffer solution for maintaining strong alkalinity, and AHMT that is reacted with formaldehyde produced when sarcosine is oxidized and serves as a chromogen and drying the result.

There is provided a method of producing a prostate cancer diagnostic test strip including: immersing a cellulose test strip in a solution containing a sarcosine oxidase producing sarcosine metabolites, a buffer solution for maintaining strong alkalinity, and MBTH that is reacted with formaldehyde produced when sarcosine is oxidized and serves as a chromogen, and drying the result.

Preferably, a water-soluble polymeric fixture for fixing added reagents may be further included.

Preferably, ethylenediaminetetraacetic acid (EDTA) for chelating metal ions in order to prevent activity of the sarcosine oxidase from being inhibited due to the metal ions may be further included.

Preferably, the sarcosine oxidase may have a concentration ranging from 200 to 500 units/dL.

Preferably, the buffer solution may have a molar concentration of 1.0 to 2.0 and a pH ranging from 9.0 to 12.5.

Preferably, the solution may further contain tartrazine for visualizing color development of a chromogen.

There is provided a method of diagnosing prostate cancer using the above prostate cancer diagnostic test strip.

According to another aspect of the present invention, there are provided a prostate cancer diagnostic test strip using peroxide, which is a sarcosine metabolite, a method of producing the same, and a method of diagnosing prostate cancer using the same. There is provided a prostate cancer diagnostic test strip including: a sarcosine oxidase producing sarcosine metabolites; a peroxide chromogen that is reacted with peroxide among the sarcosine metabolites and serves as a chromogen; and a peroxidase serving as a catalyst.

More preferably, the peroxide chromogen may be at least one of 3,3′-diaminobenzidine; 3,3′,5,5′-tetramethylbenzidine; 1,4-diaminobenzene; 1,2-dihydroxybenzene; 4-chloronaphthol; 3-amino-9-ethylcarbazole; 2,7′-diaminofluorene; N,N′-dimethylethylenediamine; and N,N′-bis-(4-aminophenyl)-1,3-xylylenediamine.

Preferably, a water-soluble polymeric fixture for fixing added reagents may be further included.

Preferably, ethylenediaminetetraacetic acid (EDTA) for chelating and removing metal ions in order to prevent activity of the sarcosine oxidase from being inhibited due to the metal ions may be further included.

There is provided a method of producing a prostate cancer diagnostic test strip, including dissolving a sarcosine oxidase and a peroxidase in a buffer solution whose pH remains in a range of 7.5 to 10.0, immersing a cellulose test strip, and then performing drying.

More preferably, the sarcosine oxidase may have a concentration ranging from 200 to 500 units/dL.

Preferably, the solution may further contain a water-soluble polymeric fixture for fixing reagents.

Preferably, the solution may further contain a dye for visualizing color development of a chromogen.

Preferably, the solution may further contain EDTA for chelating metal ions in order to prevent activity of the sarcosine oxidase from being inhibited due to the metal ions.

There is provided a method of diagnosing prostate cancer using the prostate cancer diagnostic test strip.

### Advantageous Effects

In a prostate cancer diagnostic test strip using formaldehyde, which is a sarcosine metabolite, and a method of diagnosing prostate cancer using the same according to the present invention, a diagnostic indicator of prostate cancer can be easily used without professional staff or an expensive device, unlike an HPLC method and a spectrophotometer method of the related art in which, when sarcosine in urine is qualified and quantified, a professional mixes a specimen and a reagent to cause a chemical reaction, and then analyzes the mixture using HPCL or the spectrophotometer in a laboratory.

### DESCRIPTION OF DRAWINGS

FIG. 1 is a configuration diagram illustrating an exterior of a test strip according to examples of the present invention.

FIG. 2 is a diagram showing a change in activities of a sarcosine oxidase according to a pH in a first example of the present invention.

FIG. 3 is a picture of experiment results obtained when the first example of the present invention is implemented and

FIG. 4 is an illustrated diagram thereof.

FIG. 5 is a diagram showing a change in activities of a sarcosine oxidase according to a pH in a second example of the present invention.

FIG. 6 shows a picture of experiment results obtained when a second example of the present invention is implemented and

FIG. 7 is an illustrated diagram thereof.

### MODES OF THE INVENTION

A prostate cancer diagnostic test strip using formaldehyde, which is a sarcosine metabolite, and a method of diagnosing prostate cancer using the same according to examples of the present invention will be described in detail with reference to the accompanying drawings. While the invention can be modified in various ways and take on various alternative forms, specific embodiments thereof are shown in the drawings and will herein be described in detail. It should be understood, however, that there is no intent to limit the invention to the particular forms disclosed, but on the contrary, the invention is to cover all modifications, equivalents, and alternatives falling within the spirit and scope of the invention. Like numbers refer to like elements throughout the description of the figures. In the appended drawings, structures are illustrated to have dimensions that are larger than those of actual structures for clarity of the invention or are smaller than those of actual structures for understanding schematic configurations.

Also, it will be understood that, although the terms first, second, etc. may be used herein in reference to elements of the invention, such elements should not be construed as limited by these terms. These terms are only used to distinguish one element from another. For example, a first element could be termed a second element, and a second element could be termed a first element, without departing from the scope of the present invention. Meanwhile, unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

First and second examples of the present invention will be described below in detail with reference to the accompanying drawings.

First, in a prostate cancer diagnostic test strip using formaldehyde, which is a sarcosine metabolite, and a method of diagnosing prostate cancer using the same according to the first example of the present invention, a diagnostic indicator of prostate cancer can be easily used without an expensive device or professional staff.

The fact that a concentration of sarcosine in urine unusually increases in prostate cancer patients was reported in Nature (volume 457 in 2009).

Sarcosine is decomposed by a sarcosine oxidase to form glycine, a peroxide (H₂O₂) and formaldehyde. By quantitatively detecting the peroxide and formaldehyde which are produced as metabolites of the sarcosine, it is possible to calculate a concentration of the sarcosine. Accordingly, the result can be used as a diagnostic indicator of prostate cancer. Such a metabolic process is shown in the following Chemical Formula 1.

Sarcosine+O₂+H₂O→Glycine+H₂O₂+Formaldehyde (by Sarcosine Oxidase) Chemical Formula 1

In a reaction formula shown in the following Chemical Formula 2, 4-amino-3-hydrazino-5-mercapto-1,2,4-triazole[1750-12-5] (hereinafter referred to as “AHMT”) is used. This chemical formula is used to quantify formaldehyde.

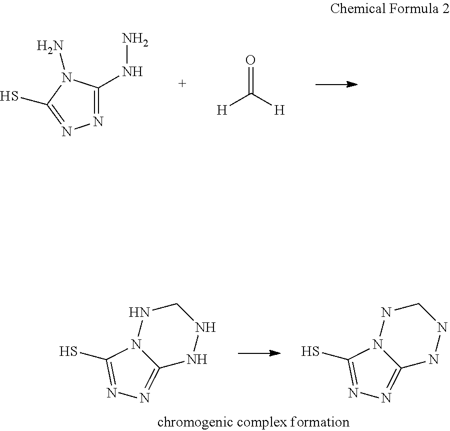 [SMILES: NNc1nnc(S)n1N ; C=O ; Sc1nnc2n1NCNN2 ; Sc1nnc2n1NCNN2]

Also, in a reaction formula shown in the following Chemical Formula 3, N-methylbenzothiazolinone-2-hydrazone (hereinafter referred to as “MBTH”) is used. This chemical formula is used to quantify formaldehyde.

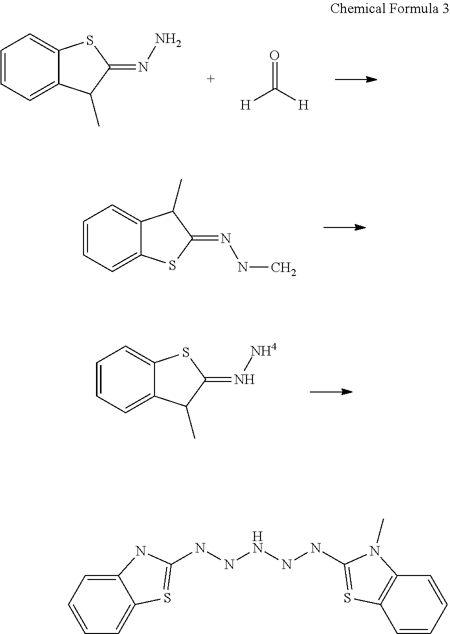

As shown in Chemical Formula 1, the peroxide and formaldehyde are produced from sarcosine by a sarcosine oxidase in proportion to a concentration of the sarcosine. In this case, exemplary factors influencing activity of the sarcosine oxidase include a pH and a concentration of a buffer solution and a reaction temperature.

In this case, as the reaction temperature, room temperature is preferable. In the buffer solution, preferably, a concentration is a molar concentration of about 1 to 2 and a pH is 8 to 9.5.

Also, as the buffer solution, a tris buffer, a phosphate buffer, a citrate buffer, a borate buffer or the like could be used.

The formaldehyde produced using the sarcosine oxidase chemically reacts with a chromogen such as 4-amino-3-hydrazino-5-mercapto-1,2,4-triazole (hereinafter referred to as “AHMT”) as shown in Chemical Formula 2 and N-methylbenzothiazolinone-2-hydrazone (hereinafter referred to as “MBTH”) as shown in Chemical Formula 3 and forms a chromogenic complex. Such a chromogenic complex is easily identifiable with the naked eye. Therefore, when a test strip using the above reaction formula is produced, it is possible to easily diagnose prostate cancer.

In this case, when a color was not easily recognizable to the eye such as blue, a method in which a yellow dye was used to form a yellow background, and a color was developed into green or cyan such that a color development tendency thereof was more visible than that of blue was sometimes additionally used. In this manner, there are several color treatment substances for easy identification with the naked eye. Tartrazine was used in the present example.

Meanwhile, when a cellulose test strip is produced, a surfactant causing urine to be easily absorbed on the test strip and a water-soluble polymeric fixture for fixing added reagents to the cellulose test strip could be added.

A reaction between the AHMT or MBTH and the formaldehyde was caused in a strong alkaline environment.

The reaction in this case depended on a pH. As shown in FIG. 2, the reaction of Chemical Formula 2 was caused at a pH of at least 9.0 or more, and a purple complex was formed. Also, the reaction of Chemical Formula 3 was caused, and a blue complex was formed and could be identified with the naked eye through a change in color of the test strip.

In a colorimetric test strip method, in consideration of the test strip in a sample under harsh pH conditions, a borate-sodium hydroxide solution (a molar concentration of 1.5 and a pH of 9.5 to 12) was prepared and used as a buffer solution. While borate-sodium hydroxide was used in the present example, another buffer solution could be used as long as strong alkalinity was sufficiently maintained. If the pH was less than that range, no reaction was caused. If the pH was higher than that range, when the cellulose test strip was immersed thereafter, the test strip was damaged due to strong alkalinity of the solution, and immersion was disabled.

In consideration of activity of an enzyme that decreased when a sarcosine oxidase test strip was heated and dried, it is necessary to add an excessive amount of the sarcosine oxidase (200 to 500 units/dL) to the buffer solution. In this case, if an amount of the sarcosine oxidase was small, activity of the enzyme decreased during a process in which the test strip was dried, and reactivity thereof was not sufficient. When an amount added was large, a cost could increase and natural discoloration could be caused.

As shown in the following Table 1, activity of the sarcosine oxidase was greatly influenced by metal ions. Since metal ions were inevitably contained in the sample, EDTA was added at 0.01 to 0.02 g/dL to chelate the metal ions and to prevent activity of the sarcosine oxidase from decreasing due to the metal ions. In this case, when an amount of EDTA added was small, the metal ions were not sufficiently removed. On the other hand, when an amount of EDTA added was large, a color development reaction actually decreased. In the present example, EDTA4NA was used. However, another substance that can chelate the metal ions could be used. Then, AHMT serving as a chromogen was dissolved at 20 to 50 mM. In this case, when an amount of the reagent added was small, there was no difference in color development between specimens of an intermediate concentration and a high concentration. On the other hand, when an amount of the reagent added was large, discoloration was caused in a natural state, which caused a change in a product or a false positive result.

TABLE 1

| Metal chloride salts | Final concentration (mM) | Relative activity (%) |
|---|---|---|
| None | — | 100 |
| Zn²⁺ | 2 | 7 |
| Mn²⁺ | 2 | 63 |
| Co²⁺ | 2 | 52 |
| Cd²⁺ | 2 | 7 |
| Ni²⁺ | 2 | 76 |
| Fe²⁺ | 2 | 66 |
| Hg²⁺ | 2 | 0 |
| Cu²⁺ | 2 | 0 |

The cellulose test strip was immersed. Then, an excessive amount of the reagent was removed, and drying was performed at 70 to 80° C. for 30 minutes.

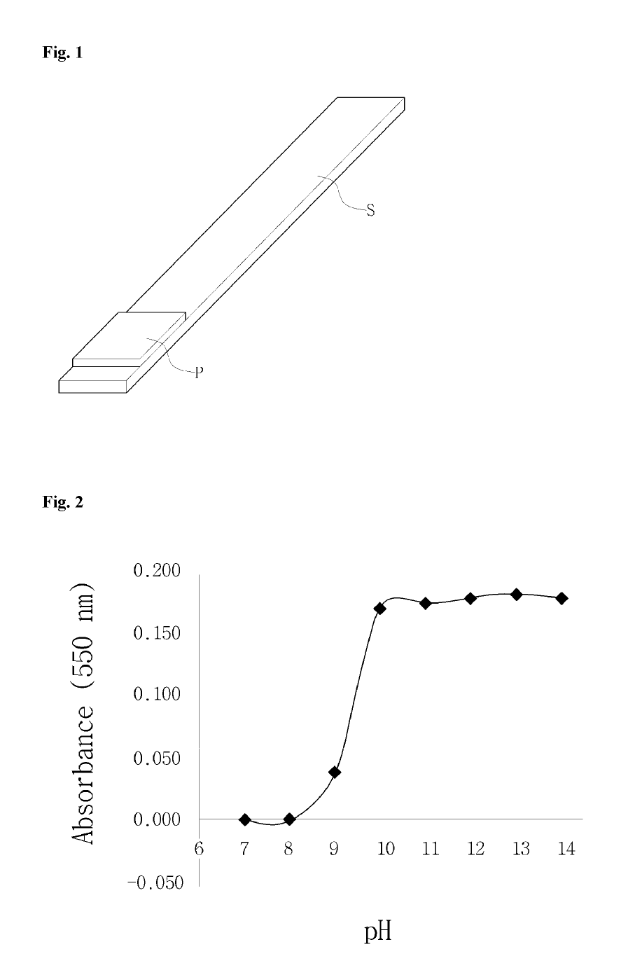

Then, a double-sided tape was attached to the test strip and cut for fixation to a support. The result was formed and prepared in the shape shown in FIG. 1. Since the result was sensitive to moisture, it was stored together with a desiccant in a container having a high level of airtightness and used as necessary.

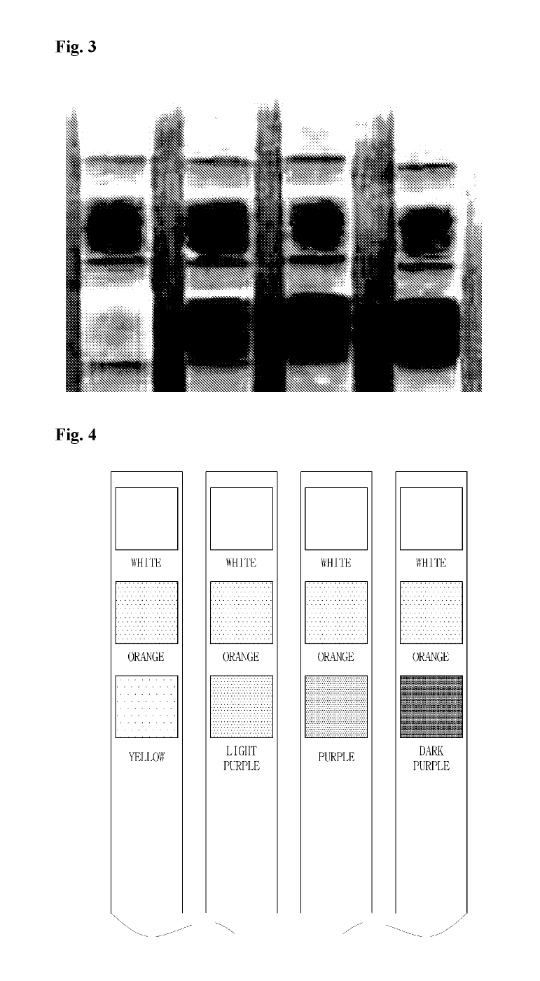

FIG. 3 shows experimental results obtained when prostate cancer is diagnosed using the test strip prepared by the above method, and FIG. 4 is an illustrated diagram thereof. In white, light orange, yellow or purple in one strip, white on the top indicates a white test strip on which no treatment was performed in order to check a background color of the sample, and the second light orange test strip is a test strip for checking a degree of dilution of urine. Pictures show sarcosine detecting reactions using formaldehyde when AHMT was used at negative, 1000, 2000, and 4000 nM on the left (test strips changed to yellow or purple)

A second example of the present invention will be described below in detail.

In a prostate cancer diagnostic test strip using a peroxide, which is a sarcosine metabolite, and a method of diagnosing prostate cancer using the same according to the second example of the present invention, a diagnostic indicator of prostate cancer can be easily used without an expensive device or professional staff.

The fact that a concentration of sarcosine in urine unusually increases in prostate cancer patients was reported in Nature (volume 457 in 2009).

That is, sarcosine is decomposed by a sarcosine oxidase to form glycine, a peroxide (H₂O₂) and formaldehyde. By quantitatively detecting the peroxide and formaldehyde which are produced as metabolites of the sarcosine, it is possible to calculate a concentration of the sarcosine. Accordingly, the result can be used as a diagnostic indicator of prostate cancer. Such a metabolic process is shown in the following Chemical Formula 4.

sarcosine oxidase sarcosine+oxygen+water->glycine+peroxide+formaldehyde Chemical Formula 4

Meanwhile, in the related art, as a method of quantifying a peroxide, a method shown in the following Chemical Formula 5 is generally known.

peroxidase peroxide+peroxide chromogen->oxidized peroxide chromogen (color)+water Chemical Formula 5

When Chemical Formula 4 and Chemical Formula 5 are used to cause reactions (represented by reaction formulae of the following Chemical Formula 6) on the test strip, a concentration of the sarcosine can be quantified through a colorimetric test strip reaction. The fundamental technological content of the present invention includes use of such a method.

sarcosine oxidase sarcosine+oxygen+water->glycine+peroxide+formaldehyde Chemical Formula 6

peroxidase peroxide+peroxide chromogen->oxidized peroxide chromogen (color)+water

As shown in Chemical Formula 4, the peroxide and formaldehyde are produced from sarcosine by a sarcosine oxidase in proportion to a concentration of the sarcosine. In this case, exemplary factors influencing activity of the sarcosine oxidase include a pH and a concentration of a buffer solution and a reaction temperature.

In this case, as the reaction temperature, room temperature is preferable. In the buffer solution, preferably, a concentration is a molar concentration of about 1 to 2 and a pH is 8 to 9.5.

Also, as the buffer solution, a tris buffer, a phosphate buffer, a citrate buffer, a borate buffer or the like could be used.

A peroxidase catalyzes a dehydrogenation reaction of the peroxide produced by the sarcosine oxidase, and a process thereof is shown in detail in Chemical Formula 6. The peroxidase is sufficiently added at an amount of about 50 to 80% of an activity level of the sarcosine oxidase. Then, a peroxide chromogen such as 3,3′-diaminobenzidine; 3,3′,5,5′-tetramethylbenzidine; 1,4-diaminobenzene; 1,2-dihydroxybenzene; 4-chloronaphthol; 3-amino-9-ethylcarbazole; 2,7′-diaminofluorene; N,N′-dimethylethylenediamine; or N,N′-bis-(4-aminophenyl)-1,3-xylylenediamine was oxidized to exhibit a predetermined color.

In this case, when a color was not easily recognizable to the eye such as blue, a method in which a yellow dye was used to form a yellow background and a color was developed into green or cyan such that a color development tendency was more visible than that of blue was sometimes additionally used.

Also, a surfactant causing urine to be easily absorbed on the test strip could be added. A fixture (a water-soluble polymer was generally used) for fixing added reagents to the cellulose test strip could be added.

Meanwhile, when two types of enzymes, the sarcosine oxidase and the peroxidase, were used at the same time, an immersion solution was separately prepared twice due to a difference of pH stabilities between the enzymes.

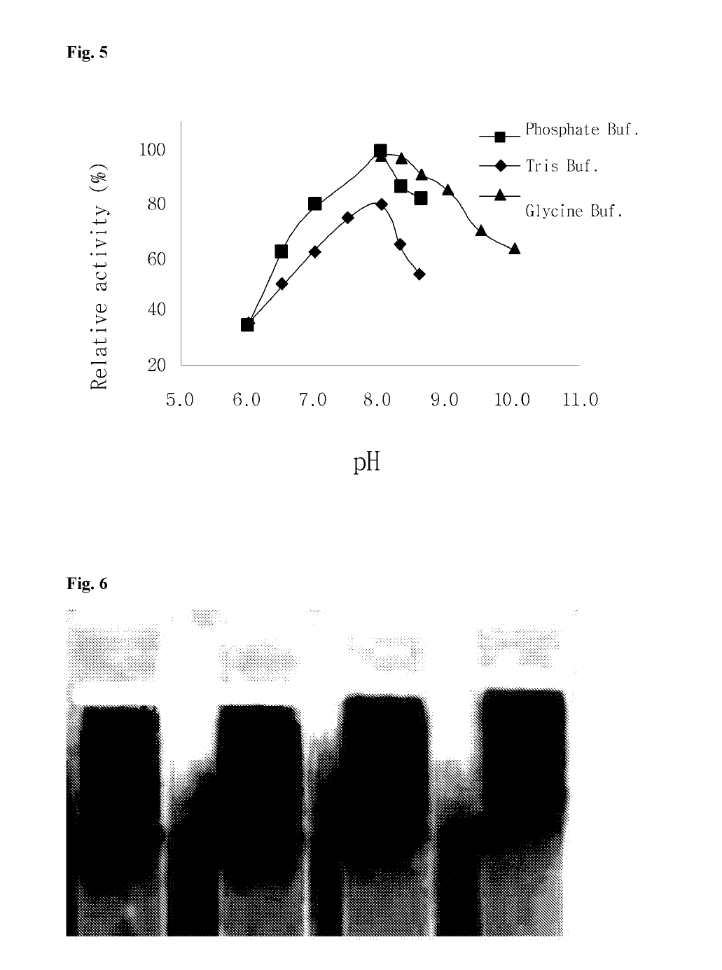

The sarcosine oxidase had maximum activity at a pH of 8.0 as shown in FIG. 5. In a general spectrophotometric method, in the buffer solution, a concentration is sufficient at a molar concentration of 0.05 to 0.1 and a pH of 7.5 to 8.5. In the present invention, the test strip prepared by the colorimetric test strip method was soaked in and taken out of the sample, and thus was exposed to the sample under harsh acidic pH conditions. In this case, in order to overcome exposure to these harsh conditions, as a buffer solution of a primary solution, a borate-sodium hydroxide solution having a molar concentration of 1.0 to 2.0 and a pH of 8.0 to 9.5 was prepared and used.

Then, in consideration of activity of an enzyme that decreased when the test strip was immersed in the prepared solution and then the test strip was heated and dried, it was necessary to add an excessive amount of the enzyme, the sarcosine oxidase, at 200 to 500 units/dL. In this case, if an amount of the sarcosine oxidase was small, activity of the enzyme decreased during a process in which the test strip was dried, and reactivity thereof was not sufficient. When an amount added was large, a cost could increase and natural discoloration could be caused.

A water-soluble polymer, a polyvinylpyrrolidone, was added at 1.0 to 2.0 g/dL in order to fix added reagents to the cellulose test strip. In this case, when an amount added was small, the reagents were not fixed to the test strip. Therefore, when the completed test strip was soaked in the sample, the reagents were released and caused color contamination. When an amount added was large, the water-soluble polymer interfered with absorption of the sample.

When 3,3′,5,5′-tetramethylbenzidine was used as a chromogen, a color was developed into a blue series. In light blue, it was not easy to identify a difference thereof with the naked eye. For this reason, tartrazine was added at 0.05 to 0.1 g/dL for easy identification with the naked eye such that, when yellow was set as a background color and a color was developed into a blue series, the yellow background and blue were mixed, and a color was developed into a green series. In this case, when an amount of tartrazine used was small, a color was developed into a blue series rather than green, due to dominance of the original color of the sample. When an amount of tartrazine used was large, a light blue reaction due to a small amount of sarcosine was blocked by dark yellow, which caused a false negative result.

As shown in the following Table 2, activity of the sarcosine oxidase is greatly influenced by metal ions. When an excessive amount of metal ions was contained in the sample, EDTA was added at 0.01 to 0.02 g/dL in order to chelate and remove the metal ions and prevent activity of the sarcosine oxidase from decreasing due to the metal ions. That is, EDTA serves as a metal adsorber and facilitates activation of the enzymes. In the present example, EDTA4NA was used. However, another substance that can chelate the metal ions could be used. When an amount of EDTA added was small, the metal ions were not sufficiently removed. On the other hand, when an amount of EDTA added was large, a color development reaction actually decreased.

TABLE 2

| Metal chloride salts | Final concentration (mM) | Relative activity (%) |
|---|---|---|
| None | — | 100 |
| Zn²⁺ | 2 | 7 |
| Mn²⁺ | 2 | 63 |
| Co²⁺ | 2 | 52 |
| Cd²⁺ | 2 | 7 |
| Ni²⁺ | 2 | 76 |
| Fe²⁺ | 2 | 66 |
| Hg²⁺ | 2 | 0 |
| Cu²⁺ | 2 | 0 |

When the reagent was sufficiently dissolved, the cellulose test strip was immersed, an excessive amount of the reagent was removed, and then drying was performed at 70 to 80° C. for 30 minutes.

In a secondary solution, in order to stably fix a peroxidase, based on a borate-hydrochloric acid buffer solution having a molar concentration of 0.5 to 1.0 and a pH of 6.0 to 7.0, the peroxidase at 100 to 200 units/dL, and 3,3′,5,5′-tetramethylbenzidine at 20 mM concentration were dissolved. In this case, when an amount of the reagent added was small, there was no difference in color development between specimens of an intermediate concentration and a high concentration. On the other hand, when an amount of the reagent added was large, discoloration was caused in a natural state, which caused a change in a product or a false positive result.

When the reagent was sufficiently dissolved, the test strip that was previously immersed in the primary solution and then dried was immersed again in the secondary solution and dried at 0 to 80° C. for 30 minutes.

Then, a double-sided tape was attached to the test strip and cut for fixation to a support. The result was formed and prepared in the shape shown in FIG. 1. Since the result was sensitive to moisture, it was stored together with a desiccant in a container having a high level of airtightness and used as necessary.

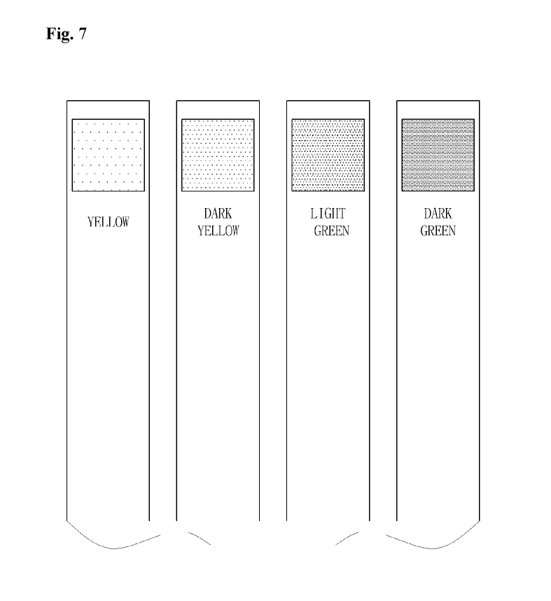

FIG. 6 shows experimental results obtained when prostate cancer is diagnosed using the test strip prepared by the above method. FIG. 7 is an illustrated diagram thereof for clarifying the results and a picture showing sarcosine detecting reactions using a peroxidase when 3,3′,5,5′-tetramethylbenzidine was used at negative, 1000, 2000, and 4000 nM on the left.

While exemplary examples of the present invention have been described above, various changes, alternations, and equivalents can be used in the present invention. It will be clear that the examples of the present invention can be appropriately changed and applied in the same manner. Therefore, the above described content does not limit the scope of the present invention defined by the accompanying claims.

1. A prostate cancer diagnostic test strip comprising:
a sarcosine oxidase producing sarcosine metabolites; and
4-amino-3-hydrazino-5-mercapto-1,2,4-triazole (AHMT) that is reacted with formaldehyde produced when sarcosine is oxidized and serves as a chromogen.

2. A prostate cancer diagnostic test strip comprising:
a sarcosine oxidase producing sarcosine metabolites; and
N-methylbenzothiazolinone-2-hydrazone (MBTH) that is reacted with formaldehyde produced when sarcosine is oxidized and serves as a chromogen.

3. The prostate cancer diagnostic test strip of claim 1, further comprising
a water-soluble polymeric fixture for fixing added reagents.

4. The prostate cancer diagnostic test strip of claim 1, further comprising
ethylenediaminetetraacetic acid (EDTA) for chelating and removing metal ions in order to prevent activity of the sarcosine oxidase from being inhibited due to the metal ions.

5. A method of producing a prostate cancer diagnostic test strip comprising:
immersing a cellulose test strip in a solution containing a sarcosine oxidase producing sarcosine metabolites, a buffer solution for maintaining strong alkalinity, and AHMT that is reacted with formaldehyde produced when sarcosine is oxidized and serves as a chromogen and drying the result.

6. A method of producing a prostate cancer diagnostic test strip comprising:
immersing a cellulose test strip in a solution containing a sarcosine oxidase producing sarcosine metabolites, a buffer solution for maintaining strong alkalinity, and MBTH that is reacted with formaldehyde produced when sarcosine is oxidized and serves as a chromogen, and drying the result.

7. The method of producing a prostate cancer diagnostic test strip of claim 5,
wherein the sarcosine oxidase has a concentration ranging from 200 to 500 units/dL.

8. The method of producing a prostate cancer diagnostic test strip of claim 5,
wherein the buffer solution has a molar concentration of 1.0 to 2.0 and has a pH ranging from 9.0 to 12.5.

9. The method of producing a prostate cancer diagnostic test strip of claim 5,
wherein the solution further contains a water-soluble polymeric fixture for fixing reagents.

10. The method of producing a prostate cancer diagnostic test strip of claim 5,
wherein the solution further contains tartrazine for visualizing color development of the chromogen.

11. The method of producing a prostate cancer diagnostic test strip of claim 5,
wherein the solution further contains EDTA for chelating metal ions in order to prevent activity of the sarcosine oxidase from being inhibited due to the metal ions.

12. A method of diagnosing prostate cancer using the prostate cancer diagnostic test strip according to any of claim 1.

13. A prostate cancer diagnostic test strip comprising:
a sarcosine oxidase producing sarcosine metabolites;
a peroxide chromogen that is reacted with peroxide among the sarcosine metabolites and serves as a chromogen; and
a peroxidase serving as a catalyst.

14. The prostate cancer diagnostic test strip of claim 13,
wherein the peroxide chromogen is at least one of 3,3′-diaminobenzidine; 3,3′,5,5′-tetramethylbenzidine; 1,4-diaminobenzene; 1,2-dihydroxybenzene; 4-chloronaphthol; 3-amino-9-ethylcarbazole; 2,7′-diaminofluorene; N,N′-dimethylethylenediamine; and N,N′-bis-(4-aminophenyl)-1,3-xylylenediamine.

15. The prostate cancer diagnostic test strip of claim 13, further comprising
a water-soluble polymeric fixture for fixing added reagents.

16. The prostate cancer diagnostic test strip of claim 13, comprising
ethylenediaminetetraacetic acid (EDTA) for chelating and removing metal ions in order to prevent activity of the sarcosine oxidase from being inhibited due to the metal ions.

17. A method of producing a prostate cancer diagnostic test strip, comprising
dissolving a sarcosine oxidase and a peroxidase in a buffer solution whose pH remains in a range of 7.5 to 10.0, immersing a cellulose test strip, and then performing drying.

18. The method of producing a prostate cancer diagnostic test strip of claim 17,
wherein the sarcosine oxidase has a concentration ranging from 200 to 500 units/dL.

19. The method of producing a prostate cancer diagnostic test strip of claim 17,
wherein the solution further contains a water-soluble polymeric fixture for fixing reagents.

20. The method of producing a prostate cancer diagnostic test strip of claim 17,
wherein the solution further contains a dye for visualizing color development of a chromogen.

21. The method of producing a prostate cancer diagnostic test strip of claim 17,
wherein the solution further contains EDTA for chelating metal ions in order to prevent activity of the sarcosine oxidase from being inhibited due to the metal ions.

22. A method of diagnosing prostate cancer using the prostate cancer diagnostic test strip according to of claim 13.
